(19) United States
(12) Patent Application Publication
(10) Pub. No.: US 2013/0142767 A1
(43) Pub. Date: Jun. 6, 2013

(54) COMPOSITION CONTAINING REDUCED COENZYME Q10, AND MANUFACTURING AND STABILISING METHODS THEREFOR

(71) Applicants: Takao Yamaguchi, Hyogo (JP); Shiro Kitamura, Hyogo (JP); Yasuyoshi Ueda, Hyogo (JP)

(72) Inventors: Takao Yamaguchi, Hyogo (JP); Shiro Kitamura, Hyogo (JP); Yasuyoshi Ueda, Hyogo (JP)

(73) Assignee: Keneka Corporation, Osaka-shi, Osaka (JP)

(21) Appl. No.: 13/642,375

(22) PCT Filed: Apr. 20, 2011
(86) PCT No.: PCT/JP2011/059762

§ 371 Date: Dec. 11, 2012

(30) Foreign Application Priority Data
Apr. 20, 2010 (JP) 2010-097263

(51) Int. Cl. A61K 31/122; A23L 1/29; A61K 8/35

(52) U.S. Cl. CPC A61K 31/122; A61K 8/355; A23L 1/29
USPC 424/94.1; 514/690; 426/648

(57) ABSTRACT

The present invention relates to a method of producing reduced coenzyme Q10, including reducing oxidized coenzyme Q10 using a reducing agent in terpenes that can highly dissolve oxidized coenzyme Q10 and reduced coenzyme Q10 in the co-existence of at least one kind of additive selected from the group consisting of alcohols, water, a surfactant and diacylglycerol. In addition, the present invention relates to a composition comprising terpenes, a reducing agent, reduced coenzyme Q10 and at least one kind selected from the group consisting of alcohols, water, a surfactant and diacylglycerol, and a method of stabilizing reduced coenzyme Q10 comprising preparing the composition.

### TECHNICAL FIELD

The present invention relates to a composition comprising reduced coenzyme Q10, a production method thereof and a stabilizing method thereof. As compared to oxidized coenzyme Q10, reduced coenzyme Q10 shows high oral absorbability, and is a compound useful as a superior food, health food, food with nutrient function claims, food for specified health use, supplement, nutritional supplement, nutritional product, drink, feed, animal drug, cosmetic, pharmaceutical product, therapeutic drug, prophylactic drug and the like.

### BACKGROUND ART

A method of obtaining reduced coenzyme Q10 as a composition in the form of a high concentration solution is important from the aspects of easy ingestion, easy preparation design and reduction of production cost of reduced coenzyme Q10. Therefore, various researches have been made to obtain a solvent capable of dissolving reduced coenzyme Q10 at high concentrations and, for example, it has been reported that limonene can dissolve reduced coenzyme Q10 at a high concentration (patent document 1 and patent document 2). Patent document 1 discloses a method of delivering coenzyme Q10 into the body by dissolving same in limonene and a composition therefor, in an attempt to more efficiently ingest coenzyme Q10. The concentration of coenzyme Q10 in the composition described in the Example of patent document 1 is 17.9 wt % at maximum. In addition, patent document 2 discloses α-lipoic acid as a stabilizer to stably maintain reduced coenzyme Q10, and fatty acid and limonene as components to be co-present. The concentration of reduced coenzyme Q10 in the composition described in patent document 2 is 23.5 wt % at maximum. Thus, the use of limonene as a solvent to dissolve reduced coenzyme Q10 enables production of a preparation containing a high concentration of reduced coenzyme Q10.

On the other hand, it is known that reduced coenzyme Q10 can be obtained by reducing oxidized coenzyme Q10. For example, it is known that oxidized coenzyme Q10 can be reduced into reduced coenzyme Q10 by using a conventional reducing agent such as sodium borohydride, sodium dithonite (sodium hydrosulfite) and the like.

### DOCUMENT LIST

### Patent Documents

patent document 1: US2005/0070611
patent document 2: WO2008/113066

### Problems to be Solved by the Invention

Based on the above-mentioned background art, the present inventors have studied various methods for reducing oxidized coenzyme Q10 in limonene as methods for producing a composition containing a high concentration of reduced coenzyme Q10. However, it has been found that a reduction reaction of oxidized coenzyme Q10 does not proceed easily in limonene even when the reaction is carried out using a known reducing agent under general reaction conditions, and a composition containing reduced coenzyme Q10 with high quality is difficult to obtain by this method by using oxidized coenzyme Q10 as a starting material. Limonene is one kind of terpenes, and other terpenes were also studied in the same manner. However, it has been found that a reduction reaction of oxidized coenzyme Q10 does not proceed easily and a composition containing reduced coenzyme Q10 with high quality is difficult to obtain.

Thus, in an attempt to conveniently produce a composition containing a high concentration of reduced coenzyme Q10, the present invention aims to provide a new production method enabling a reduction reaction of oxidized coenzyme Q10 to proceed well in terpenes that can highly dissolve reduced coenzyme Q10, a reduced coenzyme Q10 containing composition obtained by the production method, and further, a method of stabilizing reduced coenzyme Q10 utilizing the same.

### Means of Solving the Problems

The inventors have conducted intensive studies and found that, when oxidized coenzyme Q10 is reduced in terpenes capable of dissolving reduced coenzyme Q10 at a high concentration, the reduction reaction of oxidized coenzyme Q10 proceeds well even in terpenes by adding a particular additive, and reduced coenzyme Q10 can stably exist in the obtained composition, which resulted in the completion of the present invention.

Accordingly, the present invention relates to a method of producing reduced coenzyme Q10, comprising reducing oxidized coenzyme Q10 using a reducing agent in terpenes in the co-existence of at least one kind of additive selected from the group consisting of alcohols, water, a surfactant and diacylglycerol. In addition, the present invention relates to a composition containing at least one kind selected from the group consisting of alcohols, water, a surfactant and diacylglycerol, terpenes, a reducing agent and reduced coenzyme Q10, and further, a method of stabilizing reduced coenzyme Q10, comprising placing at least one kind selected from the group consisting of alcohols, water, a surfactant and diacylglycerol in co-existence with terpenes and a reducing agent.

Accordingly, the present invention provides the following.
[1] A method of producing reduced coenzyme Q10 comprising reducing oxidized coenzyme Q10 with a reducing agent in terpenes in the co-existence of at least one kind of additive selected from the group consisting of alcohols, water, a surfactant and diacylglycerol.
[2] The production method of [1], wherein the alcohol is a monovalent alcohol having 1 to 4 carbon atoms or a divalent alcohol having 2 to 4 carbon atoms.
[3] The production method of [1] or [2], wherein the alcohol is ethanol.
[4] The production method of [1], wherein the surfactant is at least one selected from the group consisting of glycerol fatty acid ester, sucrose fatty acid ester, organic acid monoglyceride, sorbitan fatty acid ester, polyoxyethylene sorbitan fatty acid ester, propylene glycol fatty acid ester, polyglycerol condensed ricinoleic acid ester, saponin and phospholipid.
[5] The production method of [1], wherein the terpene is at least one selected from the group consisting of hemiterpene, monoterpene, sesquiterpene, diterpene, sesterterpene and triterpene.
[6] The production method of [1], wherein the reducing agent is at least one selected from the group consisting of L-ascorbic acid, D-arabo-ascorbic acid, L-ascorbylpalmitate, L-ascorbylstearate, sodium borohydride, sodium hydrosulfite, retinal, an acerola extract, a pine bark extract, a KOKIYO extract, a gardenia pigment and a KOZU (fragrant vinegar).
[7] The production method of any one of [1]-[6], wherein the reduction reaction is performed in co-existence with fats, oils or mixtures thereof.
[8] The production method of [7], wherein the fats, oils or mixtures thereof is at least one kind selected from the group consisting of coconut oil, palm oil, palm kernel oil, flaxseed oil, camellia oil, brown rice germ oil, olive oil, rape seed oil, rice oil, peanuts oil, corn oil, wheat germ oil, soybean oil, perilla oil, cottonseed oil, sunflower kerel oil, kapok oil, evening primrose oil, Shea butter, sal butter, cacao butter, sesame oil, safflower oil, avocado oil, poppy-seed oil, GOBOUSHI oil, lard, milk fat, fish oil, beef fat, fats and oils obtained by processing these by fractionation, hydrogenation, transesterification and the like, medium-chain triglyceride and partial glycerides of fatty acid.
[9] The production method of any one of [1]-[8], wherein the concentration of oxidized coenzyme Q10 is 0.001 wt % or more relative to total amount of the reaction mixture at the time of the start of the reduction reaction.
[10] The production method of any one of [1]-[9], wherein the reduction reaction is performed in a preparation.
[11] The production method of [10], wherein the preparation is a capsule.
[12] The production method of [11], wherein the capsule is a soft capsule.
[13] The production method of any one of [1]-[12], wherein the reduction reaction is performed under a deoxygenated atmosphere.

[14] A composition containing terpenes, a reducing agent, reduced coenzyme Q10 and at least one kind selected from the group consisting of alcohols, water, a surfactant and diacylglycerol.
[15] The composition of [14], wherein the alcohol is a monovalent alcohol having 1 to 4 carbon atoms or a divalent alcohol having 2 to 4 carbon atoms.
[16] The composition of [14], wherein the surfactant is at least one selected from the group consisting of glycerol fatty acid ester, sucrose fatty acid ester, organic acid monoglyceride, sorbitan fatty acid ester, polyoxyethylene sorbitan fatty acid ester, propylene glycol fatty acid ester, polyglycerol condensed ricinoleic acid ester, saponin and phospholipid.
[17] The composition of [14], wherein the terpene is at least one selected from the group consisting of hemiterpene, monoterpene, sesquiterpene, diterpene, sesterterpene and triterpene.
[18] The composition of [14], wherein the reducing agent is at least one selected from the group consisting of L-ascorbic acid, D-arabo-ascorbic acid, L-ascorbylpalmitate, L-ascorbylstearate, sodium borohydride, sodium hydrosulfite, retinal, an acerola extract, a pine bark extract, a KOKIYO extract, a gardenia pigment and a KOZU (fragrant vinegar).
[19] The composition of any one of [14]-[18], further comprising fats, oils or mixtures thereof.
[20] The composition of any one of [14]-[19], wherein the content of reduced coenzyme Q10 in the composition is 0.001 wt % or more.
[21] The composition of any one of [14]-[20], wherein the reduced coenzyme Q10 is added from the outside.
[22] The composition of any one of [14]-[20], wherein the reduced coenzyme Q10 is reduced in the composition.
[23] A method of stabilizing reduced coenzyme Q10, comprising placing terpenes, a reducing agent and at least one kind selected from the group consisting of alcohols, water, a surfactant and diacylglycerol in co-existence.
[24] The stabilizing method of [23], wherein the alcohol is a monovalent alcohol having 1 to 4 carbon atoms or a divalent alcohol having 2 to 4 carbon atoms.
[25] The stabilizing method of [23], wherein the surfactant is at least one selected from the group consisting of glycerol fatty acid ester, sucrose fatty acid ester, organic acid monoglyceride, sorbitan fatty acid ester, polyoxyethylene sorbitan fatty acid ester, propylene glycol fatty acid ester, polyglycerol condensed ricinoleic acid ester, saponin and phospholipid.
[26] The stabilizing method of [23], wherein the terpene is at least one selected from the group consisting of hemiterpene, monoterpene, sesquiterpene, diterpene, sesterterpene and triterpene.
[27] The stabilizing method of [23], wherein the reducing agent is at least one selected from the group consisting of L-ascorbic acid, D-arabo-ascorbic acid, L-ascorbylpalmitate, L-ascorbylstearate, sodium borohydride, sodium hydrosulfite, retinal, an acerola extract, a pine bark extract, a KOKIYO extract, a gardenia pigment and a KOZU (fragrant vinegar).
[28] The stabilizing method of any one of [23]-[27], further comprising fats, oils or mixtures thereof in co-existence.
[29] The stabilizing method of any one of [23]-[28], wherein reduced coenzyme Q10 is added from the outside.
[30] The stabilizing method of any one of [23]-[29], wherein the co-existence is formed under a deoxygenated atmosphere.

### Effect of the Invention

According to the present invention, oxidized coenzyme Q10 and reduced coenzyme Q10 can be dissolved at high concentrations by using terpenes in combination with at least one kind of additive selected from the group consisting of alcohols, water, a surfactant and diacylglycerol, as well as oxidized coenzyme Q10 can be rapidly reduced into reduced coenzyme Q10 in said solution. Therefore, a convenient method of producing a reduced coenzyme Q10 preparation and a composition containing a high concentration of reduced coenzyme Q10 can be provided. Furthermore, a method of stabilizing easily oxidizable reduced coenzyme Q10 can also be provided.

### DESCRIPTION OF EMBODIMENTS

The present invention is explained in detail in the following.

First, the method of producing reduced coenzyme Q10 of the present invention is explained. The production method of the present invention is characterized by reduction of oxidized coenzyme Q10 using a reducing agent in terpenes in the co-existence of at least one kind of additive selected from the group consisting of alcohols, water, a surfactant and diacylglycerol.

Oxidized coenzyme Q10 to be a starting material in the production method of the present invention may be oxidized coenzyme Q10 alone or a mixture thereof with reduced coenzyme Q10. When the above-mentioned oxidized coenzyme Q10 is a mixture thereof with reduced coenzyme Q10, the ratio of oxidized coenzyme Q10 in the total amount of coenzyme Q10 (i.e., total amount of reduced coenzyme Q10 and oxidized coenzyme Q10) is not particularly limited and is, for example, not less than 1 wt %, normally not less than 5 wt %, preferably not less than 10 wt %, more preferably not less than 20 wt %, particularly preferably not less than 50 wt %. While the upper limit is not particularly limited, when a mixture with oxidized coenzyme Q10 and reduced coenzyme Q10 is used as a starting material, it is normally not more than 99.9 wt %. Needless to say, oxidized coenzyme Q10 alone can be used. Oxidized coenzyme Q10 to be used here can be obtained by a conventionally known method, for example, synthesis, fermentation, extraction from a naturally-occurring substance and the like. Preferably, it is obtained by fermentation or extraction from a naturally-occurring substance.

In the production method of the present invention, terpenes capable of dissolving oxidized coenzyme Q10 and reduced coenzyme Q10 at high concentrations are used as reaction solvents for reducing oxidized coenzyme Q10, and obtaining reduced coenzyme Q10.

The terpenes to be used as reaction solvents for a reduction reaction in the production method of the present invention are not particularly limited, and all of hemiterpene, monoterpene, sesquiterpene, diterpene, sesterterpene and triterpene can be used preferably. Among these, hemiterpene, monoterpene and sesquiterpene are more preferable, monoterpene and sesquiterpene are particularly preferable, monoterpene is most preferable, in view of the ability to dissolve oxidized coenzyme Q10 and reduced coenzyme Q10.

Specific examples of the terpenes usable for the production method of the present invention include prenol, 3-methyl-3-buten-2-ol, tiglic acid, angelic acid, senecioic acid, isovaleric acid, alloocimene, β-bourbonene, δ-cadinene, dehydro-p-cymene, menthol, dl-limonene, d-limonene, l-limonene, p-cymene, α-pinene, valencene, myrcene, bisabolene, carene, caryophyllene, terpinene, phytol, cis-3,7-dimethyl-1,3,6-octatriene, δ-elemene, β-elemene, α-farnesene, β-farnesene, farnesene, germacrene D, β-guaiene, longifolene, β-ocimene, α-phellandrene, pinocamphone, sabinene, terpinolen, thujopsis, α-copaene, hydrogenated limonene dimer, isocaryophyllene, pinene dimer, dipentene dimer, dipentene trimer, geraniol, citral, citronellal, citronellol, 1,8-cineol, hydroxycitronellal, linalool, cosmene, nerol, myrcenol, lavandulol, ipsdienol, neral, geranial, perylene, rose furan, geranyl acid, thioterpineol, α-terpineol, β-terpineol, γ-terpineol, δ-terpineol, carveol, terpin, perillaldehyde, perillalcohol, carvone, ascaridole, anethole, thujone, thujanol, α-ionone, β-ionone, γ-ionone, farnesol, nerolidol, α-sinensal, β-sinensal, bisabol, squalene, citronellyl oxyacetoaldehyde, myrtenal, perillaldehyde, 2-p-cymenol, 2-ethoxy-p-cymene, carvenol, 4-carvomenthenol, carvyl acetate, carvyl propionate, caryophyllene alcohol, caryophyllenealcohol acetate, 1,4-cineol, eugenol, d-selinene, thymol, d-camphene, linalool acetate and the like.

Preferred are prenol, 3-methyl-3-buten-2-ol, tiglic acid, angelic acid, senecioic acid, isovaleric acid, alloocimene, β-bourbonene, δ-cadinene, dehydro-p-cymene, dl-limonene, d-limonene, 1-limonene, p-cymene, α-pinene, valencene, myrcene, bisabolene, carene, caryophyllene, terpinene, phytol, cis-3,7-dimethyl-1,3,6-octatriene, δ-elemene, β-elemene, α-farnesene, β-farnesene, farnesene, germacrene D, β-guaiene, longifolene, β-ocimene, α-phellandrene, pinocamphone, sabinene, thujopsis, α-copaene, hydrogenated limonene dimer, isocaryophyllene, pinene dimer, dipentene dimer, dipentene trimer, geraniol, citral, citronellal, citronellol, 1,8-cineol, hydroxycitronellal, linalool, nerol, myrcenol, neral, geranial, carvone, anethole, thujone, squalene, eugenol, d-selinene, thymol or linalool acetate, more preferably δ-cadinene, dl-limonene, d-limonene, l-limonene, p-cymene, α-pinene, valencene, myrcene, bisabolene, carene, caryophyllene, terpinene, phytol, α-phellandrene, geraniol, citral, citronellol, 1,8-cineol, linalool, carvone, anethole, thujone, eugenol, d-selinene, thymol or linalool acetate, particularly preferably monoterpene such as dl-limonene, d-limonene, l-limonene, p-cymene, α-pinene, myrcene, carene, terpinene, α-phellandrene, geraniol, citral, citronellol, 1,8-cineol, linalool, carvone, anethole, thujone, thymol, linalool acetate and the like or bisabolene, and most preferred are dl-limonene and d-limonene.

In addition, an essential oil containing the above-mentioned terpenes can also be used as terpenes in the present invention. While the essential oil usable in this case is not particularly limited as long as it contains terpenes, orange oil, capsicum oil, mustard oil, garlic oil, callaway oil, clove oil, cinnamon oil, cocoa extract, coffee bean extract, ginger oil, spearmint oil, celery-seed oil, thyme oil, onion oil, nutmeg oil, parsley seed oil, mint oil, vanilla extract, fennel oil, pennyroyal oil, peppermint oil, eucalyptus oil, lemon oil, rose oil, rosemary oil, almond oil, ajowan oil, anise oil, amyris oil, angelica root oil, ambrette seed oil, estragon oil, origanum oil, orris root oil, olibanum oil, cassia oil, cascarilla oil, cananga oil, chamomile oil, calamus oil, cardamom oil, carrot seed oil, cubeb oil, cumin oil, grapefruit oil, cinnamon leaf oil, cade oil, pepper oil, costus root oil, congnac oil, copaiba oil, cilantro oil, perilla oil, musk, juniper berry oil, star anis oil, sage oil, savory oil, geranium oil, tangerin oil, dill oil, neroli oil, tolu balsam oil, basil oil, birch oil, patchouli oil, palmarosa oil, pimento oil, petitgrain oil, bay leaf oil, bergamot oil, peru balsam oil, benzoin resin, Bois de Rose oil, hop oil, boronia absolute, marjoram oil, mandarin oil, myrtle oil, Chinese lemon flavor, lime oil, lavandin oil, lavender oil, rue oil, lemongrass oil, lenthionine, lavage oil, laurel leaf oil, worm wood oil and the like can be mentioned.

In the production method of the present invention, any of alcohols, water, a surfactant and diacylglycerol, or a mixture thereof is used as an additive for promoting the reduction reaction of oxidized coenzyme Q10 in terpenes (hereinafter to be referred to as “the additive of the present invention”).

The alcohols usable as the additive of the present invention are not particularly limited and may be cyclic or acyclic, or saturated or unsaturated. Generally, alcohols having 1 to 20 carbon atoms can be mentioned, of which those having 1 to 12 carbon atoms are preferable, 1 to 5 carbon atoms are more preferable, and 1 to 4 carbon atoms are particularly preferable. Among these, a monovalent alcohol is preferable. Most preferred is a monovalent alcohol having 2 carbon atoms. In addition, divalent alcohols having 2 to 5 carbon atoms, preferably 2 to 4 carbon atoms, more preferably 3 carbon atoms, and trivalent alcohols having 3 carbon atoms are also preferably used.

Examples of the monovalent alcohol include methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-undecanol, 1-dodecanol, allyl alcohol, propargyl alcohol, benzyl alcohol, cyclohexanol, 1-methylcyclohexanol, 2-methylcyclohexanol, 3-methylcyclohexanol, 4-methylcyclohexanol, cinnamyl alcohol, phenol, α-methylbenzyl alcohol and the like.

Preferred are methanol, ethanol, 1-propanol, 2-propanol, 1-butanol, 2-butanol, isobutylalcohol, tert-butyl alcohol, 1-pentanol, 2-pentanol, 3-pentanol, 2-methyl-1-butanol, isopentylalcohol, tert-pentylalcohol, 3-methyl-2-butanol, neopentylalcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-dodecanol, benzyl alcohol, cyclohexanol, cinnamylalcohol, phenol and α-methylbenzyl alcohol, more preferred are methanol, ethanol, 1-propanol, 2-propanol, 1-butanol, 2-butanol, isobutylalcohol, 1-pentanol, 2-pentanol, 2-methyl-1-butanol, isopentylalcohol, 3-methyl-2-butanol, 1-hexanol, 4-methyl-2-pentanol, 1-heptanol, 2-heptanol, 3-heptanol, 1-octanol, 2-octanol, 2-ethyl-1-hexanol, 1-nonanol, 1-decanol, 1-dodecanol, benzyl alcohol, cyclohexanol, cinnamylalcohol, phenol and α-methylbenzyl alcohol, still more preferred are methanol, ethanol, 1-propanol, 2-propanol, 1-butanol, 2-butanol, isobutylalcohol, 2-methyl-1-butanol and isopentylalcohol, particularly preferred are methanol, ethanol, 2-propanol and 1-butanol, and most preferred is ethanol.

Examples of the divalent alcohol include 1,2-ethanediol, 1,2-propanediol(propylene glycol), 1,3-propanediol, 1,2-butanediol, 1,3-butanediol, 1,4-butanediol, 2,3-butanediol, 1,5-pentanediol and the like. Preferred are 1,2-ethanediol, 1,2-propanediol, 1,3-butanediol and 1,3-propanediol, and most preferred is 1,2-propanediol.

As the trivalent alcohol, glycerol and the like can be preferably used.

When optical isomers exist for the aforementioned alcohols, any thereof or a mixture thereof can also be used. For example, an L form, D form or a racemate which is a mixture thereof can also be used.

The surfactant usable as the additive in the present invention is not particularly limited and, for example, glycerol fatty acid ester, sucrose fatty acid ester, organic acid monoglyceride, sorbitan fatty acid ester, polyoxyethylene sorbitan fatty acid ester, propylene glycol fatty acid ester, polyglycerol condensed ricinoleic acid ester (condensed ricinoleic acid glyceride), saponin, phospholipid and the like can be mentioned.

While glycerol fatty acid ester is not particularly limited, for example, glycerols having a polymerization degree of 1-10 can be mentioned. In addition, glycerol fatty acid ester wherein each fatty acid has 6 to 18 carbon atoms and the like can be mentioned. While the fatty acid residue constituting glycerol fatty acid ester is not particularly limited, fatty acid having 6 to 18 carbon atoms can be preferably used and, for example, caprylic acid, capric acid, lauric acid, myristic acid, palmitic acid, stearic acid, isostearic acid, oleic acid, linoleic acid, linolenic acid and the like can be mentioned.

Examples of the sucrose fatty acid ester include, but are not limited to, one wherein fatty acid having 6 to 22 carbon atoms is ester-bonded to one or more hydroxyl groups of sucrose, such as sucrose laurate, sucrose myristate, sucrose palmitate, sucrose stearate, sucrose oleate, sucrose behenate, sucrose erucate and the like.

While the organic acid monoglyceride is not particularly limited, succinic acid monoglyceride such as monoglycerol caprylic acid succinic acid ester, monoglycerol stearic acid succinic acid ester and the like; citric acid monoglyceride such as monoglycerol oleic acid citric acid ester, monoglycerol stearic acid citric acid ester and the like; acetic acid monoglyceride such as monoglycerol stearic acid acetic acid ester and the like; monoglycerol stearic acid lactic acid ester, monoglycerol stearic acid diacetyltartaric acid ester and the like can be mentioned.

Examples of the sorbitan fatty acid ester include, but are not limited to, sorbitan wherein one or more of the hydroxyl groups are ester bonded to fatty acid each having 6 to 18 carbon atoms, such as sorbitan monolaurate, sorbitan monopalmitate, sorbitan monostearate, sorbitan monooleate and the like.

Examples of the polyoxyethylene sorbitan fatty acid ester include, but are not limited to, sorbitan polyoxyethylene monopalmitate, sorbitan polyoxyethylene monostearate, sorbitan polyoxyethylene monooleate, sorbitan polyoxyethylene tristearate and sorbitan polyoxyethylene trioleate, wherein 6-20 mol of an ethyleneoxide chain is added, and the like.

As the propylene glycol fatty acid ester, any of monoester or diester can be used. While the fatty acid residue constituting the propylene glycol fatty acid ester is not particularly limited, fatty acid having 6 to 18 carbon atoms can be preferably used, and for example, caprylic acid, capric acid, lauric acid, myristic acid, palmitic acid, stearic acid, isostearic acid, oleic acid, linoleic acid, linolenic acid and the like can be mentioned.

Examples of the polyglycerol condensed ricinoleic acid ester include, but are not limited to, one wherein polyglycerol has an average polymerization degree of 2 to 10, and polyricinoleic acid has an average condensation degree (average number of condensed ricinoleic acid) of 2 to 4, such as tetraglycerol condensed ricinoleate, pentaglycerol condensed ricinoleate, hexaglycerol condensed ricinoleate and the like.

Examples of the saponin include, but are not limited to, enju saponin, quillaja saponin, purification soybean saponin, yucca saponin and the like.

Examples of the phospholipid include, but are not limited to, lecithin such as egg-yolk lecithin, purified soybean lecithin and the like, phosphatidylcholine, phosphatidylethanolamine, phosphatidylserine, sphingomyelin, dicetyl phosphoric acid, stearylamine, phosphatidylglycerol, phosphatidic acid, phosphatidylinositolamine, cardiolipin, ceramide phosphoryl ethanolamine, ceramide phosphoryl glycerol and a mixture thereof and the like. Needless to say, a phospholipid after processing of hydrogenation, enzyme degradation and the like (hydrogenated lecithin and lysolecithin) can also be used. To improve absorbability of reduced coenzyme Q10, an enzymatically degraded phospholipid (lysolecithin) is also preferably used.

In the production method of the present invention, glycerol fatty acid ester, sucrose fatty acid ester, organic acid monoglyceride, sorbitan fatty acid ester, propylene glycol fatty acid ester and phospholipid are preferable, and phospholipid is particularly preferable, from among the above-mentioned surfactants. Specifically, polyglycerol fatty acid esters such as decaglycerol pentaolate, tetraglycerol monolaurate, decaglycerol monomyristate, decaglycerol monooleate, diglycerol monooleate and the like; monoglycerol fatty acid esters such as glycerol monostearate, glycerol monooleate and the like; sucrose oleate, acetic acid monoglycerides such as monoglycerol lauric acid acetic acid ester and the like, citric acid monoglyceride such as monoglycerol oleic acid citric acid ester and the like, sorbitan monooleate, propyleneglycol monooleate, propyleneglycol monostearate, lecithin and the like are particularly preferable.

Diacylglycerol usable as the additive in the present invention may be any of 1,2-diacylglycerol and 1,3-diacylglycerol or a mixture thereof as long as two fatty acid groups in glycerol fatty acid esters are bonded. In addition, the fatty acid group is not particularly limited and, for example, any fatty acid having 8 to 22 carbon atoms can be selected. Furthermore, the fatty acid composition thereof may be single or mixture. Moreover, commercially available diacylglycerol obtained by hydrolyzing one fatty acid of vegetable oil and the like can also be preferably used.

Moreover, in the production method of the present invention, other solvents other than the above-mentioned terpenes and the additives of the present invention can be used in combination as long as no seriously adverse influence is exerted on the progress of the reduction reaction of oxidized coenzyme Q10. While other solvents to be used in the production method of the present invention are not particularly limited, organic solvents such as hydrocarbons, fatty acid esters, ethers, ketones, nitrogen compounds (including nitriles, amides), sulfur compounds and the like, fatty acids, fats and oils can be mentioned.

While the above-mentioned hydrocarbons are not particularly limited, for example, aliphatic hydrocarbon, aromatic hydrocarbon, halogenated hydrocarbon and the like can be mentioned. Particularly, aliphatic hydrocarbon and aromatic hydrocarbon are preferable, and aliphatic hydrocarbon is especially preferable.

While aliphatic hydrocarbon may be cyclic or acyclic, saturated or unsaturated and is not particularly limited, acyclic aliphatic hydrocarbon is particularly preferably used. In addition, aliphatic hydrocarbon having 3 to 20 carbon atoms, preferably 5 to 12 carbon atoms, can be generally used.

Specific examples of the aliphatic hydrocarbon include propane, butane, isobutane, pentane, 2-methylbutane, cyclopentane, 2-pentene, hexane, 2-methylpentane, 2,2-dimethylbutane, 2,3-dimethylbutane, methylcyclopentane, cyclohexane, 1-hexene, cyclohexene, heptane, 2-methylhexane, 3-methylhexane, 2, 3-dimethylpentane, 2,4-dimethylpentane, methylcyclohexane, 1-heptene, octane, 2,2,3-trimethylpentane, isooctane, ethylcyclohexane, 1-octene, nonane, 2,2,5-trimethylhexane, 1-nonene, decane, 1-decene, p-menthane, undecane, dodecane and the like.

Among these, saturated aliphatic hydrocarbon having 5 to 8 carbon atoms is preferable, and pentane, 2-methylbutane, cyclopentane, hexane, 2-methylpentane, 2,2-dimethylbutane, 2,3-dimethylbutane, methylcyclopentane, cyclohexane, heptane, 2-methylhexane, 3-methylhexane, 2,3-dimethylpentane, 2,4-dimethylpentane, methylcyclohexane, octane, 2,2,3-trimethylpentane, isooctane, ethylcyclohexane and the like are particularly preferable.

While the aromatic hydrocarbon is not particularly limited, normally, aromatic hydrocarbon having 6 to 20 carbon atoms, particularly 6 to 12 carbon atoms, especially 7 to 10 carbon atoms, is preferably used. Specific examples of the aromatic hydrocarbon include benzene, toluene, xylene, o-xylene, m-xylene, p-xylene, ethylbenzene, cumene, mesitylene, tetralin, butylbenzene, p-cymene, cyclohexylbenzene, diethylbenzene, pentylbenzene, dipentylbenzene, dodecylbenzene, styrene and the like. It is preferably toluene, xylene, o-xylene, m-xylene, p-xylene, ethylbenzene, cumene, mesitylene, tetralin, butylbenzene, p-cymene, cyclohexylbenzene, diethylbenzene or pentylbenzene, more preferably, toluene, xylene, o-xylene, m-xylene, p-xylene, cumene or tetralin, and most preferably cumene.

The halogenated hydrocarbon may be cyclic or acyclic, saturated or unsaturated, and is not particularly limited. In general, acyclic one is preferably used. Normally, chlorinated hydrocarbon and fluorinated hydrocarbon are preferable, and chlorinated hydrocarbon is particularly preferable. A halogenated hydrocarbon having 1 to 6 carbon atoms, particularly 1 to 4 carbon atoms, especially 1 or 2 carbon atoms, is preferably used.

Specific examples of the halogenated hydrocarbon include dichloromethane, chloroform, carbon tetrachloride, 1,1-dichloroethane, 1,2-dichloroethane, 1,1,1-trichloroethane, 1,1,2-trichloroethane, 1,1,1,2-tetrachloroethane, 1,1,2,2-tetrachloroethane, pentachloroethane, hexachloroethane, 1,1-dichloroethylene, 1,2-dichloroethylene, trichloroethylene, tetrachloroethylene, 1,2-dichloropropane, 1,2,3-trichloropropane, chlorobenzene, 1,1,1,2-tetrafluoroethane and the like.

It is preferably dichloromethane, chloroform, carbon tetrachloride, 1,1-dichloroethane, 1,2-dichloroethane, 1,1,1-trichloroethane, 1,1,2-trichloroethane, 1,1-dichloroethylene, 1,2-dichloroethylene, trichloroethylene, chlorobenzene or 1,1,1,2-tetrafluoroethane, more preferably dichloromethane, chloroform, 1,2-dichloroethylene, trichloroethylene, chlorobenzene or 1,1,1,2-tetrafluoroethane.

While the above-mentioned fatty acid esters are not particularly limited, for example, formic acid ester, acetic acid ester, propionic acid ester, butyric acid ester, isovaleric acid ester, heptane acid ester, hexane acid ester and the like can be mentioned. Acetic acid ester and formic acid ester are particularly preferable, and acetic acid ester is especially preferable.

Examples of the formic acid ester include methyl formate, ethyl formate, propyl formate, isopropyl formate, butyl formate, isobutyl formate, sec-butyl formate, pentyl formate, isoamyl formate, geranyl formate, citronellyl formate and the like. Preferred are methyl formate, ethyl formate, propyl formate, isoamyl formate, geranyl formate and citronellyl formate, and most preferred is ethyl formate.

Examples of the acetic acid ester include methyl acetate, ethyl acetate, propyl acetate, isopropyl acetate, butyl acetate, isobutyl acetate, sec-butyl acetate, pentyl acetate, isopentyl acetate, isoamyl acetate, sec-hexyl acetate, cyclohexyl acetate, benzyl acetate, amyl acetate, geranyl acetate, citronellyl acetate, cinnamyl acetate, terpinyl acetate, 1-menthyl acetate, linalyl acetate and the like. Preferred are methyl acetate, ethyl acetate, isopropyl acetate, isoamyl acetate, cyclohexyl acetate, benzyl acetate, amyl acetate, geranyl acetate, citronellyl acetate, cinnamyl acetate, terpinyl acetate, 1-menthyl acetate and linalyl acetate, more preferred are ethyl acetate, isopropyl acetate and butyl acetate, and most preferred is ethyl acetate.

Examples of the propionic acid ester include methyl propionate, ethyl propionate, butyl propionate, isopentyl propionate, isoamyl propionate and the like.

Examples of the butyric acid ester include methyl butyrate, ethyl butyrate, butyl butyrate, isopentyl butyrate, isoamyl propionate, cyclohexyl butyrate and the like. Examples of the isovaleric acid ester include methyl isovalerate, ethyl isovalerate, butyl isovalerate, isopentyl isovalerate, isoamyl isovalerate, allyl isovalerate and the like.

Examples of the heptane acid ester include methyl heptanoate, ethyl heptanoate, butyl heptanoate, isopentyl heptanoate, allyl heptanoate, and the like. Examples of the hexane acid ester include methyl hexanoate, ethyl hexanoate, butyl hexanoate, isopentyl hexanoate, allyl hexanoate and the like.

The above-mentioned ethers may be cyclic or acyclic, or saturated or unsaturated, and are not particularly limited. Generally, saturated ones are preferably used.

Specific examples of the ethers include diethyl ether, methyl tert-butyl ether, dipropyl ether, diisopropyl ether, dibutyl ether, dihexyl ether, ethylvinyl ether, butylvinyl ether, anisole, phenetole, butylphenyl ether, methoxytoluene, dioxane, furan, 2-methylfuran, tetrahydrofuran, tetrahydropyran, ethylene glycol dimethyl ether, ethylene glycol diethyl ether, ethylene glycol dibutyl ether, ethylene glycol monomethyl ether, ethylene glycol monoethyl ether, ethylene glycol dibutyl ether, dibenzyl ether, benzyl butyl ether and the like.

Preferred are diethyl ether, methyl tert-butyl ether, dipropyl ether, diisopropyl ether, dibutyl ether, dihexyl ether, anisole, phenetole, butylphenyl ether, methoxytoluene, dioxane, 2-methylfuran, tetrahydrofuran, tetrahydropyran, ethylene glycol dimethyl ether, ethylene glycol diethyl ether, ethylene glycol dibutyl ether, ethylene glycol monomethyl ether, ethylene glycol monoethyl ether, dibenzyl ether and benzyl butyl ether, more preferred are diethyl ether, methyl tert-butyl ether, diisopropyl ether, anisole, dioxane, tetrahydrofuran, ethylene glycol monomethyl ether, ethylene glycol monoethyl ether, dibenzyl ether and benzyl butyl ether, still more preferred are diethyl ether, diisopropyl ether, dibenzyl ether, benzyl butyl ether and the like, and most preferred is diisopropyl ether.

The above-mentioned ketones may be cyclic or acyclic, or saturated or unsaturated, and are not particularly limited. Specific examples include acetone, methyl ethyl ketone, methyl butyl ketone, methyl isobutyl ketone, acetophenone, 4-methoxyphenyl acetone, paramethyl acetophenone, methyl β-naphthl ketone and the like. Preferred are paramethyl acetophenone and methylethyl ketone, and most preferred is methylethylketone.

The above-mentioned nitriles may be cyclic or acyclic, or saturated or unsaturated, and are not particularly limited. In general, saturated ones are preferably used. Normally, nitrile having 2 to 20 carbon atoms, particularly 2 to 12 carbon atoms, especially 2 to 8 carbon atoms, is preferably used. Specific examples of the nitriles include acetonitrile, propionitrile, malononitrile, butyronitrile, isobutyronitrile, succinonitrile, valeronitrile, glutaronitrile, hexanenitrile, heptyl cyanide, octyl cyanide, undecanenitrile, dodecanenitrile, tridecanenitrile, pentadecanenitrile, stearonitrile, chloroacetonitrile, bromoacetonitrile, chloropropionitrile, bromopropionitrile, methoxyacetonitrile, methyl cyanoacetate, ethyl cyanoacetate, tolunitrile, benzonitrile, chlorobenzonitrile, bromobenzonitrile, cyanobenzoic acid, nitrobenzonitrile, anisonitrile, phthalonitrile, bromotolunitrile, methylcyanobenzoate, methoxybenzonitrile, acetylbenzonitrile, naphtonitrile, biphenylcarbonitrile, phenylpropionitrile, phenylbutyronitrile, methylphenylacetonitrile, diphenylacetonitrile, naphthylacetonitrile, nitrophenylacetonitrile, chlorobenzyl cyanide, cyclopropanecarbonitrile, cyclohexanecarbonitrile, cycloheptanecarbonitrile, phenylcyclohexanecarbonitrile, tolylcyclohexanecarbonitrile and the like.

Preferred are acetonitrile, propionitrile, succinonitrile, butyronitrile, isobutyronitrile, valeronitrile, methyl cyanoacetate, ethyl cyanoacetate, benzonitrile, tolunitrile or chloropropionitrile, more preferred are acetonitrile, propionitrile, butyronitrile and isobutyronitrile, and most preferred is acetonitrile.

Examples of the nitrogen compounds other than the aforementioned nitriles include nitromethane, triethylamine, pyridine, formamide, N-methylformamide, N,N-dimethylformamide, N,N-dimethylacetamide, N-methylpyrrolidone and the like.

Examples of the sulfur compounds include dimethyl sulfoxide, sulfolane and the like.

The above-mentioned fatty acids may be cyclic or acyclic, saturated or unsaturated, fatty carboxylic acid or aromatic carboxylic acid, or with or without a functional group, and any fatty acid can be used. Preferred are monovalent fatty carboxylic acid, which is specifically formic acid, acetic acid, propionic acid, butyric acid, valeric acid, caproic acid, enanthic acid, glycolic acid, lactic acid, glycerol acid, hydroxybutyric acid, leucine acid, mevalonic acid, pantoic acid, ricinoleic acid, ricinelaidic acid, cerebronic acid, quinic acid, shikimic acid, gluconic acid, sorbic acid, pantothenic acid, caprylic acid, capric acid, lauric acid, myristic acid, palmitic acid, stearic acid, isostearic acid, oleic acid, linoleic acid, linolenic acid, arachidonic acid, behenic acid, eicosapentaenoic acid, docosahexaenoic acid, docosapentaenoic acid or the like.

In the production method of the present invention, fats, oils or mixture thereof may also be further copresent during reduction. The above-mentioned fat, oil or mixture thereof may be natural fat or oil from animal or vegetable, synthetic fat or oil, or processed fat or oil. Examples of the vegetable fat or vegetable oil include coconut oil, palm oil, palm kernel oil, flaxseed oil, camellia oil, brown rice germ oil, rape seed oil, rice oil, peanuts oil, corn oil, wheat germ oil, soybean oil, perilla oil, cottonseed oil, sunflower seed oil, kapok oil, evening primrose oil, Shea butter, sal butter, cacao butter, sesame oil, safflower oil, olive oil, avocado oil, poppyseed oil, burdock fruit oil and the like. Examples of the animal fat and animal oil include lard, milk fat, fish oil, beef fat and the like. Furthermore, fat or oil (e.g., hydrogenated oil) obtained by processing the above by fractionation, hydrogenation, transesterification and the like can also be mentioned. Needless to say, medium-chain triglyceride (MCT), partial glycerides of fatty acid and the like can also be used. In addition, a mixture thereof may be used.

The medium-chain triglyceride is not particularly limited and, for example, triglyceride wherein fatty acid has 6 to 12 carbon atoms, preferably 8 to 12 carbon atoms, and the like can be mentioned.

Of the above-mentioned fats, oils or mixtures thereof, vegetable fat, vegetable oil, synthetic fat, synthetic oil, processed fat, and processed oil are preferable from the aspects of easy handling, odor and the like. Among these, specifically, coconut oil, palm oil, palm kernel oil, rape seed oil, rice oil, soybean oil, cottonseed oil, safflower oil, olive oil, medium-chain triglyceride (MCT), partial triglyceride of fatty acid and the like are preferable, and rice oil, soybean oil, rape seed oil, safflower oil, medium-chain triglyceride, partial triglycerides of fatty acid and the like are particularly preferable.

While the above-mentioned other solvents can be preferably used for the present invention, those acceptable to foods, pharmaceutical products, cosmetics and the like are preferable, and solvents acceptable for foods are more preferable. Since a reaction product can be directly ingested without processing, fats and oils are particularly preferable.

The reducing agent to be used for the production method of the present invention is not particularly limited as long as it can convert oxidized coenzyme Q10 to reduced coenzyme Q10. For example, even naturally-derived components such as an extract or pigment from animal or plant, a product of animal or plant and the like, and even compounds can be used as a reducing agent in the production method of the present invention even when they are not conventionally used as reducing agents, as long as they have an ability to convert oxidized coenzyme Q10 to reduced coenzyme Q10.

Examples of the extract from animal or plant, which is usable as a reducing agent in the production method of the present invention, include acerola extract, pine bark extract, KOKIYO extract, tea extract (green tea extract, sweet tea extract, oolong tea extract and the like), DOKUDAMI (Houttuynia cordata) extract, enzyme-treated rutin, BENIKOJI (Monascus purpureus) extract, cranberry extract, rosemary extract, ampelopsis extract, Maca extract, Indian Dates extract and the like. Among these, acerola extract, pine bark extract, KOKIYO extract, DOKUDAMI (Houttuynia cordata) extract and enzyme-treated rutin are preferable, and acerola extract, pine bark extract and KOKIYO extract are particularly preferable.

Examples of the pigment derived from animal or plant, which is usable as a reducing agent in the production method of the present invention, include cacao pigment, gardenia pigment, grape peel pigment, BENIKOJI (Monascus purpureus) pigment and the like. Among these, gardenia pigment is preferable.

Examples of the product of animal or plant usable as a reducing agent in the production method of the present invention include royal jelly, KOZU (fragrant vinegar) and the like, and KOZU (fragrant vinegar) is more preferable.

Examples of the compound usable as a reducing agent in the production method of the present invention include L-ascorbic acid, D-arabo-ascorbic acid, L-ascorbylpalmitate, L-ascorbylstearate, sodium borohydride, sodium hydrosulfite, retinal, β-carotene, tocotrienol, NADH, cyanocobalamin, octyl gallate, dodecyl gallate, sesamol, thiamine hydrochloride and the like. Among these, L-ascorbic acid, D-arabo-ascorbic acid, L-ascorbylpalmitate, L-ascorbylstearate, sodium borohydride, sodium hydrosulfite, retinal, β-carotene and tocotrienol are preferable, L-ascorbic acid, D-arabo-ascorbic acid, L-ascorbylpalmitate, L-ascorbylstearate, sodium borohydride, sodium hydrosulfite and retinal are particularly preferable, L-ascorbic acid and L-ascorbylpalmitate are still more preferable, and L-ascorbylpalmitate is most preferable.

In the production method of the present invention, the reduction reaction only requires co-existence of oxidized coenzyme Q10 and a reducing agent, which are the starting materials, in the existence of, where necessary, the above-mentioned terpenes and the additive of the present invention, and the method therefor is not limited. The co-existence in this case only requires contact of oxidized coenzyme Q10 and a reducing agent, which are the starting materials, in the reaction system, wherein the system is not particularly limited and may be uniform or non-uniform. Needless to say, a system with high contact efficiency of oxidized coenzyme Q10 and a reducing agent is effective for reducing oxidized coenzyme Q10. From this aspect, it is most preferable that oxidized coenzyme Q10 and a reducing agent be present in the same liquid phase.

In the production method of the present invention, the concentration of the additive relative to the reaction system at the time of start of the reduction reaction (total weight of reaction mixture) is not particularly limited. It is normally about 0.1 wt % or more, preferably about 1 wt % or more, more preferably about 5 wt % or more, particularly preferably about 10 wt % or more, further preferably about 20 wt % or more, still more preferably about 30 wt % or more, and particularly about 50 wt % or more. While the upper limit of the concentration of the additive relative to the reaction system at the time of start of the reduction reaction (total weight of reaction mixture) is not particularly limited, it is normally about 99 wt % or less, preferably about 90 wt % or less, more preferably about 80 wt % or less, particularly preferably about 60 wt % or less, so as to secure the content of reduced coenzyme Q10 itself and in view of the dissolution of oxidized coenzyme Q10.

In the production method of the present invention, the method of addition of the additive to the reaction system is not particularly limited, and the additive may be added alone to the reaction system, or terpenes and/or other solvent containing these additives may be used as a reaction solvent. Furthermore, oxidized coenzyme Q10 and a reducing agent containing these additives can also be used. Moreover, the form of additive at the time of addition is not particularly limited, and the additive may be in any form of solid, liquid or gas. Needless to say, it is desirable to uniformly mix the additive with terpenes to be the reaction solvents, for effective function of the additive in the reaction system.

Therefore, it is preferable to add the additive itself in a liquid state or as a solution of additive dissolved in terpenes, other solvent and the like.

In the production method of the present invention, the concentration of the terpenes relative to the reaction system at the time of start of the reduction reaction (total weight of reaction mixture) is not particularly limited. It is normally about 1 wt % or more, preferably about 5 wt % or more, more preferably about 10 wt % or more, particularly preferably about 20 wt % or more, further preferably about 30 wt % or more, and still more preferably about 40 wt % or more. While the upper limit of the concentration of the terpenes relative to the reaction system at the time of start of the reduction reaction is not particularly limited, it is normally about 99 wt % or less, preferably about 90 wt % or less, more preferably about 80 wt % or less, particularly preferably about 70 wt % or less, so as to secure the content of reduced coenzyme Q10 itself and in view of the dissolution of oxidized coenzyme Q10.

In the production method of the present invention, while the weight ratio of oxidized coenzyme Q10 and the reducing agent at the time of start of the reaction is not particularly limited, generally, it is normally about 1/1000 or more, preferably about 1/100 or more, more preferably about 1/10 or more, particularly preferably about 1/1 or more, as the weight ratio of the reducing agent to oxidized coenzyme Q10 (reducing agent/oxidized coenzyme Q10). While the upper limit of the weight ratio of the reducing agent to oxidized coenzyme Q10 is not particularly limited, it is about 10000/1 or less, preferably about 1000/1 or less, more preferably about 100/1 or less, particularly preferably about 10/1 or less, from the aspects of economic aspect and the effectiveness of the obtained composition as a nutrient.

In the production method of the present invention, the concentration of oxidized coenzyme Q10 relative to the reaction system at the time of start of the reduction reaction (the total weight of the whole reaction mixture) is not particularly limited. It is generally about 0.001 wt %, preferably about 0.01 wt % or more, more preferably about 0.1 wt % or more, still more preferably about 0.2 wt % or more, particularly preferably about 1 wt % or more, further more preferably about 2 wt % or more, and among these, about 3 wt % or more. While the upper limit thereof is not particularly limited, it is normally 99 wt % or less, preferably 98 wt % or less, more preferably 96 wt % or less.

While the reaction temperature of the reduction reaction in the production method of the present invention is not particularly limited, the reaction is performed generally at 20° C. or more, preferably 30° C. or more, more preferably 40° C. or more, further preferably 50° C. or more, particularly preferably 60° C. or more. While the upper limit of the reaction temperature is not particularly limited, it is normally 200° C. or lower, preferably 150° C. or lower, more preferably 120° C. or lower.

To exhibit the effect of the present invention at the maximum, the above-mentioned reduction reaction is preferably performed, for example, under a deoxygenation atmosphere. The deoxygenation atmosphere can be formed by inert gas replacement, reducing pressure, boiling and combining them. At least, inert gas replacement, that is, use of inert gas atmosphere, is preferable. Examples of the above-mentioned inert gas include nitrogen gas, helium gas, argon gas, hydrogen gas, carbon dioxide gas and the like, preferably nitrogen gas.

In the production method of the present invention, the reduction reaction can also be performed in the preparation. That is, a production method of reduced coenzyme Q10 comprising preparing a composition containing oxidized coenzyme Q10, a reducing agent, terpenes and the additive of the present invention, processing the mixture into a preparation form, and reducing oxidized coenzyme Q10 to reduced coenzyme Q10 in the preparation is within the scope of the present invention. The reduction in this case is performed over a predetermined period by preservation, heating and the like. In the present invention, the preparation means an oral administration form such as capsule (hard capsule, soft capsule, microcapsule), tablet, syrup, drink and the like, or a form such as cream, suppository, toothpaste and the like. The preparation in which the reduction reaction is performed is preferably the above-mentioned oral administration form, which is more preferably capsule and particularly preferably soft capsule.

Reduced coenzyme Q10 can be conveniently produced by the production method of the present invention as mentioned above. Here, the ratio of reduced coenzyme Q10 to the total amount of coenzyme Q10 at the time of completion of the reaction (i.e., total amount of reduced coenzyme Q10 and oxidized coenzyme Q10) is normally about 65 wt % or more, preferably about 70 wt % or more, more preferably 85 wt % or more, particularly preferably 90 wt % or more, especially 95 wt % or more, most of all 98 wt % or more.

The composition and stabilizing method of the present invention are explained in the following.

The composition of the present invention is a composition of reduced coenzyme Q10, which contains terpenes, a reducing agent, reduced coenzyme Q10 and at least one kind selected from the group consisting of alcohols, water, a surfactant and diacylglycerol. The stabilizing method of the present invention is a method of stabilizing reduced coenzyme Q10 by placing terpenes, a reducing agent and at least one kind selected from the group consisting of alcohols, water, a surfactant and diacylglycerol in co-existence. That is, reduced coenzyme Q10 can be stabilized by forming the composition of the present invention.

In the composition and stabilizing method of the present invention, reduced coenzyme Q10 to be contained in the composition and to be the target of stabilization can be obtained by, for example, a method known per se such as synthesis, fermentation, extraction from naturally occurring substance, reduction of oxidized coenzyme Q10 and the like. Preferred are those obtained by reducing oxidized coenzyme Q10 such as existing high pure coenzyme Q10 and the like, or a mixture of oxidized coenzyme Q10 and reduced coenzyme Q10 with a general reducing agent, such as sodium hydrosulfite (sodium dithionite), sodium borohydride, ascorbic acids and the like. More preferred are those obtained by reducing oxidized coenzyme Q10 such as existing high pure coenzyme Q10 and the like, or a mixture of oxidized coenzyme Q10 and reduced coenzyme Q10 with ascorbic acids. In addition, needless to say, reduced coenzyme Q10 obtained by the above-mentioned production method of the present invention can also be used preferably.

The reduced coenzyme Q10 to be used for the composition and stabilizing method of the present invention may be reduced coenzyme Q10 alone or a mixture with oxidized coenzyme Q10. When the above-mentioned reduced coenzyme Q10 is a mixture with oxidized coenzyme Q10, the proportion of reduced coenzyme Q10 in the total amount of coenzyme Q10 (that is, total amount of reduced coenzyme Q10 and oxidized coenzyme Q10) is not particularly limited. It is normally about 65 wt % or more, preferably about 70 wt % or more, more preferably 85 wt % or more, particularly preferably 90 wt % or more, especially 95 wt % or more, most or all 98 wt % or more. While the upper limit is not particularly limited, it is normally 99.9 wt % or less.

Specific examples and detailed explanation of the reducing agent to be used for the composition and stabilizing method of the present invention are the same as those for the production method of the present invention, and L-ascorbic acid, D-arabo-ascorbic acid, L-ascorbylpalmitate, L-ascorbylstearate, sodium borohydride, sodium hydrosulfite, retinal, acerola extract, pine bark extract, KOKIYO extract, gardenia pigment, KOZU (fragrant vinegar) and the like can be used as more preferable examples.

In the composition and stabilizing method of the present invention, the weight ratio of reduced coenzyme Q10 and the reducing agent to be contained in the composition or used for stabilization is not particularly limited. It is generally about 1/1000 or more, preferably about 1/100 or more, more preferably about 1/10 or more, particularly preferably about 1/1 or more, as the weight ratio of the reducing agent to reduced coenzyme Q10 (reducing agent/reduced coenzyme). While the upper limit of the weight ratio of the reducing agent to reduced coenzyme Q10 is not particularly limited, it is about 10000/1 or less, preferably about 1000/1 or less, more preferably about 100/1 or less, particularly preferably about 10/1 or less.

The composition and stabilizing method of the present invention are characterized by the use of terpenes that can dissolve reduced coenzyme Q10 at high concentrations as solvents, and further, combined use of at least one kind selected from the group consisting of alcohols, water, a surfactant and diacylglycerol, that is, the additive of the present invention, to stably maintain reduced coenzyme Q10.

As the terpenes to be used for the composition and stabilizing method of the present invention, hemiterpene, monoterpene, sesquiterpene, diterpene, sesterterpene, triterpene and the like can be used as described for the production method of the present invention. From the aspect of dissolution ability, hemiterpene, monoterpene and sesquiterpene are more preferable, monoterpene and sesquiterpene are particularly preferable, and monoterpene is most preferable. In addition, the details and preferable examples of the terpenes usable for the composition and stabilizing method of the present invention are as described for the production method of the present invention.

In the composition and stabilizing method of the present invention, as mentioned above, the additives of the present invention such as alcohols, water, surfactant, diacylglycerol and the like are used. Specific examples and preferable examples of the additives of the present invention are as described for the production method of the present invention.

In the composition and stabilizing method of the present invention, the concentration of the additive contained in the composition is not particularly limited. It is normally about 0.1 wt % or more, preferably about 0.5 wt % or more, more preferably about 1 wt % or more, particularly preferably about 5 wt % or more, more preferably about 10 wt % or more, and among these, about 20 wt % or more, relative to the total weight of the composition. While the upper limit of the concentration of the additive relative to the total weight of the composition is not particularly limited, it is normally about 99 wt % or less, preferably about 90 wt % or less, more preferably about 80 wt % or less, particularly preferably about 60 wt % or less, from the aspect of solubility of reduced coenzyme Q10.

In the composition and stabilizing method of the present invention, the concentration of the terpenes contained in the composition is not particularly limited. It is normally about 1 wt % or more, preferably about 5 wt % or more, more preferably about 10 wt % or more, particularly preferably about 20 wt % or more, still more preferably about 30 wt % or more, moreover preferably 40 wt % or more, relative to the total weight of the composition. While the upper limit of the concentration of the terpenes to the total weight of the composition is not particularly limited, it is normally about 99 wt % or less, preferably about 90 wt % or less, more preferably about 80 wt % or less, particularly preferably about 70 wt % or less, to secure the content of reduced coenzyme Q10 itself, and from the aspects of the ability to dissolve reduced coenzyme Q10, the property of the obtained composition and the like.

In addition, other solvents can be added besides the above-mentioned terpenes and the additive of the present invention, as long as oxidation of reduced coenzyme Q10 is not promoted. The solvent to be used for the composition and stabilizing method of the present invention is not particularly limited, and examples thereof include organic solvents such as hydrocarbons, fatty acid esters, ethers, ketones, nitrogen compounds (including nitriles and amides), sulfur compounds and the like, fatty acids, fats, oils or mixtures thereof and the like. Specific examples and detailed explanation of other solvents which can be used in the composition and stabilizing method of the present invention are as described for the production method of the present invention. Fats, oils or mixtures thereof are preferable.

In the composition and stabilizing method of the present invention, reduced coenzyme Q10 and a reducing agent co-exist in the composition in the existence of the above-mentioned terpenes and the additive of the present invention. The “co-existence” here only requires that they are contacted in some form. The contact form is not particularly limited, and the composition system may be uniform or non-uniform. A system with high contact efficiency of reduced coenzyme Q10 and a reducing agent is effective for stabilization of reduced coenzyme Q10, and it is most preferable that reduced coenzyme Q10 and a reducing agent be present in the same liquid phase.

In the composition and stabilizing method of the present invention, as a substance other than reduced coenzyme Q10, a reducing agent, terpenes, the additive of the present invention and the above-mentioned other solvents, for example, excipient, disintegrant, lubricant, binder, dye, anticoagulant, absorption promoter, solubilizer, stabilizer, active ingredient other than reduced coenzyme Q10, and the like can be contained in the composition, and they are not particularly limited.

While the above-mentioned excipient is not particularly limited, for example, sucrose, lactose, glucose, starch, mannitol, crystalline cellulose, calcium phosphate, calcium sulfate and the like can be mentioned.

While the above-mentioned disintegrant is not particularly limited, for example, starch, agar, calcium citrate, calcium carbonate, crystalline cellulose, carboxymethylcellulose, tragacanth, alginic acid and the like can be mentioned.

While the above-mentioned lubricant is not particularly limited, for example, talc, magnesium stearate, polyethylene glycol, silica, hydrogenated oil and the like can be mentioned.

While the above-mentioned binder is not particularly limited, for example, ethylcellulose, methylcellulose, hydroxypropylmethylcellulose, hydroxypropylcellulose, tragacanth, shellac, gelatin, pullulan, gum arabic, polyvinylpyrrolidone, polyvinyl alcohol, polyacrylic acid, polymethacrylic acid, sorbitol and the like can be mentioned.

While the above-mentioned dye is not particularly limited, for example, dye such as titanium oxide, synthesis colors, colcothar dye, tar pigment and the like can be mentioned.

While the above-mentioned anticoagulant is not particularly limited, for example, stearic acid, talc, light anhydrous silicic acid, hydrated silicon dioxide and the like can be mentioned.

While the above-mentioned absorption promoter is not particularly limited, for example, higher alcohols, higher fatty acids and the like can be mentioned.

While the above-mentioned solubilizing agent for active ingredients is not particularly limited, for example, organic acids such as fumaric acid, succinic acid etc., and the like can be mentioned.

While the above-mentioned stabilizer is not particularly limited, for example, benzoic acid, beeswax, hydroxypropylmethylcellulose, methylcellulose and the like can be mentioned.

The above-mentioned other active ingredients except coenzyme Q10 are not particularly limited. For example, amino acids, vitamins such as vitamin C and vitamin E and derivatives thereof, carotenoids such as β-carotin and astaxanthin, minerals, polyphenols, organic acids, saccharides, peptides, proteins and the like can be mentioned.

The above-mentioned substances may have plural roles. For example, starch may have roles of excipient and disintegrant.

In the composition and stabilizing method of the present invention, the content of the reducing agent to the total weight of the composition is not particularly limited. From the aspect of sufficient exhibition of the reduced coenzyme Q10-stabilizing effect, it is normally about 0.01 wt % or more, preferably about 0.1 wt % or more, more preferably about 1 wt % or more, particularly preferably about 10 wt % or more.

In the composition and stabilizing method of the present invention, the content of reduced coenzyme Q10 to the total weight of the composition is not particularly limited. It is generally about 0.001 wt %, preferably about 0.01 wt % or more, more preferably about 0.1 wt % or more, further preferably about 0.2 wt % or more, particularly preferably about 1 wt % or more, moreover preferably about 2 wt % or more, and among these, about 3 wt % or more. In addition, from the aspect of production of a high concentration-solution composition, for example, it can be set to about 10 wt % or more, preferably about 20 wt % or more, more preferably about 23 wt % or more, further preferably 25 wt % or more, particularly preferably 30 wt % or more. While the upper limit is not particularly limited, for example, it is 90 wt % or less, preferably 80 wt % or less.

In the composition and stabilizing method of the present invention, a method of preparing a composition containing reduced coenzyme Q10, terpenes, the additive of the present invention and a reducing agent is not particularly limited. For example, when reduced coenzyme Q10 added from outside is used, reduced coenzyme Q10, a reducing agent, terpenes and the additive of the present invention may be simply mixed, or after mixing these, the aforementioned other solvents and other components may be further mixed therewith. Alternatively, a reducing agent, the additive of the present invention and other components may be mixed with a solution obtained by dissolving reduced coenzyme Q10 in terpenes.

Alternatively, reduced coenzyme Q10 obtained by the aforementioned production method of the present invention may be directly utilized. That is, a mixture wherein reduced coenzyme Q10 and a reducing agent after completion of the reduction reaction are copresent with terpenes and the additive of the present invention may be directly utilized as the composition of the present invention, and this embodiment is one of the most preferable embodiments.

To exhibit the effect of the present invention at the maximum, the stabilization method of the present invention is preferably performed in a deoxygenation atmosphere, that is, the above-mentioned co-existence is achieved, and the composition of the present invention is preferably prepared and/or preserved under a deoxygenation atmosphere. In addition, the processing into a preparation and preservation after processing to be mentioned below are also preferably performed in a deoxygenation atmosphere. The deoxygenation atmosphere can be formed by inert gas replacement, reducing pressure, boiling and combining them. At least, inert gas replacement, that is, use of inert gas atmosphere, is preferable. Examples of the above-mentioned inert gas include nitrogen gas, helium gas, argon gas, hydrogen gas, carbon dioxide gas and the like, preferably nitrogen gas.

While the composition of the present invention can be used directly, it can also be used after processing into a preparation as described for the production method of the present invention, such as oral administration forms such as capsules (hard capsule, soft capsule, microcapsule), tablets, syrups, drinks and the like, and a form for cream, suppository, toothpaste and the like. Of these, processing into the above-mentioned oral administration form is preferable. Particular preferred is the form of a capsule, particularly a soft capsule. A capsule base material in this case is not particularly limited, and gelatin derived from beef bones, cattle skin, pig skin, fish skin and the like, as well as other base materials (e.g., seaweed-derived products which are usable for food additives such as carrageenan, alginic acid and the like, viscosity increasing stabilizers of vegetable seed-derived products such as locust bean gum, guar gum and the like, and cellulose-containing agents for production material) can also be used.

In the composition of the present invention, since not only reduced coenzyme Q10, which is an active ingredient, is protected from oxidation and stably maintained, but also a preparation containing a high concentration of reduced coenzyme Q10 can be produced using terpenes, and other components effective as nutrient are also contained, the composition is expected to show a synergistic effect with reduced coenzyme Q10 and can also be a composition useful as foods and supplement such as food with nutrient function claims, food for specified health uses and the like, drinks, pharmaceutical products, animal drugs, cosmetics, pet foods and the like.

### EXAMPLES

While the present invention is explained in more detail in the following by referring to Examples, the present invention is not limited to those Examples alone.

Moreover, the weight ratio of reduced coenzyme Q10 and oxidized coenzyme Q10 in the Examples were measured by the following HPLC analysis. However, the weight ratio of reduced coenzyme Q10 and oxidized coenzyme Q10 does not define the upper limit thereof in the present invention. To conveniently indicate the weight ratio of reduced coenzyme Q10 and oxidized coenzyme Q10 in the present Example, the ratio of reduced coenzyme Q10 to the total amount of coenzyme Q10 (total amount of oxidized coenzyme Q10 and reduced coenzyme Q10) is shown in percentage as a “weight ratio of reduced coenzyme Q10”. For example, “the weight ratio of reduced coenzyme Q10 is 20%” means that the weight ratio of reduced coenzyme Q10 and oxidized coenzyme Q10 is 20/80.
(HPLC analysis conditions)
column; SYMMETRY C18 (manufactured by Waters) 250 mm (length) 4.6 mm (inner diameter),
mobile phase; C₂H₅OH:CH₃OH=4:3 (v:v),
detection wavelength; 210 nm,
flow rate; 1 ml/min,
retention time of reduced coenzyme Q10; 9.1 min,
retention time of oxidized coenzyme Q10; 13.3 min.

### Example 1

Limonene (d-limonene) and ethanol in the amounts indicated in Table 1 were added in a 25 ml test tube, and oxidized coenzyme Q10 (200 mg, 0.23mmol) and L-ascorbylpalmitate (576 mg, 6-fold equivalents) as a reducing agent were added. The vessel was substituted with nitrogen, and a reduction reaction was performed by stirring at 80° C. for 16 hr. The weight ratio of reduced coenzyme Q10 in the reaction mixture after the reaction is shown in Table 1. As a comparison, the result without addition of ethanol is also shown in Table 1.

TABLE 1

| | amount of | amount of | weight ratio (%) |
|---|---|---|---|
| | limonene added | EtOH added | of reduced |
| | (g) | (g) | coenzyme Q10 |
| comparison | 3.00 | 0.00 | 34 |
| Run. 1 | 2.97 | 0.03 | 88 |
| Run. 2 | 1.50 | 1.50 | 93 |

### Example 2

Limonene (d-limonene) and ethanol in the amounts indicated in Table 2 were added in a 25 ml test tube, and oxidized coenzyme Q10 (200 mg, 0.23 mmol) and L-ascorbic acid (245 mg, 6-fold equivalents) as a reducing agent were added. The vessel was substituted with nitrogen, and a reduction reaction was performed by stirring at 80° C. for 16 hr. The weight ratio of reduced coenzyme Q10 in the reaction mixture after the reaction is shown in Table 2. As a comparison, the result without addition of ethanol is also shown in Table 2.

TABLE 2

| | amount of | amount of EtOH | weight ratio (%) |
|---|---|---|---|
| | limonene added | added | of reduced |
| | (g) | (g) | coenzyme Q10 |
| comparison | 3.00 | 0.00 | 0 |
| Run. 1 | 1.50 | 1.50 | 70 |

### Example 3

Limonene (d-limonene) and water in the amounts indicated in Table 3 were added in a 25 ml test tube, and oxidized coenzyme Q10 (200 mg, 0.23 mmol) and L-ascorbylpalmitate (576 mg, 6-fold equivalents) as a reducing agent were added. The vessel was substituted with nitrogen, and a reduction reaction was performed by stirring at 80° C. for 16 hr. The weight ratio of reduced coenzyme Q10 in the reaction mixture after the reaction is shown in Table 3. As a comparison, the result without addition of water is also shown in Table 3.

TABLE 3

| | amount of | amount of | weight ratio (%) |
|---|---|---|---|
| | limonene added | water added | of reduced |
| | (g) | (g) | coenzyme Q10 |
| comparison | 3.00 | 0.00 | 34 |
| Run. 1 | 2.85 | 0.15 | 95 |
| Run. 2 | 2.70 | 0.30 | 95 |
| Run. 3 | 2.40 | 0.60 | 93 |

### Example 4

Limonene (d-limonene, 2.85 g) and additives such as alcohols and the like indicated in Table 4 (each 0.15 g) were added in a 25 ml test tube, and oxidized coenzyme Q10 (200 mg, 0.23 mmol) and L-ascorbylpalmitate (576 mg, 6-fold equivalents) as a reducing agent were added. The vessel was substituted with nitrogen, and a reduction reaction was performed by stirring at 80° C. for 16 hr. The weight ratio of reduced coenzyme Q10 in the reaction mixture after the reaction is shown in Table 4. As a comparison, the result of using limonene (3.00 g) and without addition of an additive is also shown in Table 4.

TABLE 4

| | | weight ratio (%) |
|---|---|---|
| | additive | of reduced coenzyme Q10 |
| comparison | without addition | 34 |
| Run. 1 | methanol | 88 |
| Run. 2 | 2-propanol | 89 |
| Run. 3 | 1-butanol | 89 |
| Run. 4 | propylene glycol | 63 |

### Example 5

Limonene (d-limonene, 1.50 g) and additives (each 1.50 g) such as surfactant, diacylglycerol and the like indicated in Table 5 were added in a 25 ml test tube, and oxidized coenzyme Q10 (200 mg, 0.23 mmol) and L-ascorbylpalmitate (576 mg, 6-fold equivalents) as a reducing agent were added. The vessel was substituted with nitrogen, and a reduction reaction was performed by stirring at 80° C. for 16 hr. The weight ratio of reduced coenzyme Q10 in the reaction mixture after the reaction is shown in Table 5. As a comparison, the result by using limonene (3.00 g) and without addition of an additive is also shown in Table 5.

TABLE 5

| | | weight ratio (%) of |
|---|---|---|
| | additive | reduced coenzyme Q10 |
| comparison | without addition | 34 |
| Run. 1 | diacylglycerol (*) | 77 |
| Run. 2 | decaglycerol pentaoleate | 80 |
| Run. 3 | tetraglycerol condensed | 77 |
| | ricinoleic acid | |
| Run. 4 | acetic acid monoglyceride | 100 |
| | (lauric) | |
| Run. 5 | propylene glycol | 82 |
| | monooleate | |
| Run. 6 | propylene glycol | 65 |
| | monostearate | |
| Run. 7 | citric acid monoglyceride | 77 |
| | (oleic) | |
| Run. 8 | tetraglycerol monolaurate | 100 |
| Run. 9 | sorbitan monooleate | 100 |
| Run. 10 | sucrose oleate | 75 |
| Run. 11 | lecithin | 100 |
| Run. 12 | glycerol monostearate | 99 |

(*) diacylglycerol: ″SANFUT GDO-D″ manufactured by TAIYO KAGAKU, fatty acid composition: mainly containing oleic acid, diacylglycerol content 96%, other monoglyceride 4%

### Example 6

Limonene (d-limonene, 1.50 g) and additives (each 1.50 g) such as alcohols, surfactant and the like indicated in Table 6 were added in a 25 ml test tube, and oxidized coenzyme Q10 (200 mg, 0.23 mmol) and a pine bark extract (main component, pycnogenol, 2.00 g, 10-fold weight) as a reducing agent were added. The vessel was substituted with nitrogen, and a reduction reaction was performed by stirring at 80° C. for 16 hr. The weight ratio of reduced coenzyme Q10 in the reaction mixture after the reaction is shown in Table 6. As a comparison, the result by using limonene (3.00 g) and without addition of an additive is also shown in Table 6.

TABLE 6

| | | weight ratio (%) of |
|---|---|---|
| | additive | reduced coenzyme Q10 |
| comparison | without addition | 30 |
| Run. 1 | ethanol | 100 |
| Run. 2 | lecithin | 100 |

### Example 7

Terpinene (1.50 g) and additives such as surfactant and the like (each 1.50 g) indicated in Table 7 were added in a 25 ml test tube, and oxidized coenzyme Q10 (200 mg, 0.23 mmol) and L-ascorbic acid (245 mg, 6-fold equivalents) as a reducing agent were added. The vessel was substituted with nitrogen, and a reduction reaction was performed by stirring at 80° C. for 16 hr. The weight ratio of reduced coenzyme Q10 in the reaction mixture after the reaction is shown in Table 7. As a comparison, the result by using terpinene (3.00 g) and without addition of an additive is also shown in Table 7.

TABLE 7

| | | weight ratio (%) of |
|---|---|---|
| | additive | reduced coenzyme Q10 |
| comparison | without addition | 4 |
| Run. 1 | tetraglycerol monolaurate | 64 |
| Run. 2 | lecithin | 99 |

### Example 8

Bisabolene (1.50 g) and additives such as alcohols, water, surfactant and the like (each 1.50 g) indicated in Table 8 were added in a 25 ml test tube, and oxidized coenzyme Q10 (200 mg, 0.23 mmol) and L-ascorbylpalmitate (576 mg, 6-fold equivalents) as a reducing agent were added. The vessel was substituted with nitrogen, and a reduction reaction was performed by stirring at 80° C. for 16 hr. The weight ratio of reduced coenzyme Q10 in the obtained reaction mixture is shown in Table 8. As a comparison, the result by using bisabolene (3.00 g) and without addition of an additive is also shown in Table 8.

TABLE 8

| | | weight ratio (%) of |
|---|---|---|
| | additive | reduced coenzyme Q10 |
| comparison | without addition | 35 |
| Run. 1 | ethanol | 76 |
| Run. 2 | water | 100 |
| Run. 3 | 1-butanol | 75 |
| Run. 4 | tetraglycerol monolaurate | 75 |
| Run. 5 | lecithin | 99 |

### Example 9

Bisabolene (1.50 g) and additives such as alcohols, surfactant and the like (each 1.50 g) indicated in Table 9 were added in a 25 ml test tube, and oxidized coenzyme Q10 (200 mg, 0.23 mmol) and L-ascorbic acid (245 mg, 6-fold equivalents) as a reducing agent were added. The vessel was substituted with nitrogen, and a reduction reaction was performed by stirring at 80° C. for 16 hr. The weight ratio of reduced coenzyme Q10 in the reaction mixture after the reaction is shown in Table 9. As a comparison, the result by using bisabolene (3.00 g) and without addition of an additive is also shown in Table 9.

TABLE 9

| | | weight ratio (%) of |
|---|---|---|
| | additive | reduced coenzyme Q10 |
| comparison | without addition | 31 |
| Run. 1 | ethanol | 79 |
| Run. 2 | tetraglycerol monolaurate | 73 |
| Run. 3 | lecithin | 100 |

### Example 10

Limonene (d-limonene, 26.5 kg) and ethanol (0.27 kg) were added in a reaction container, and oxidized coenzyme Q10 crystal (20 kg, 23.2 mol) and L-ascorbylpalmitate (1.8 equivalents, 17.3 kg, 41.8 mol) as a reducing agent were added. The reaction vessel was substituted with nitrogen, and a reduction reaction was performed by stirring at 80° C. for 24 hr to give a composition containing reduced coenzyme Q10. A gelatin soft capsule preparation directly containing the composition resulting from the reaction was produced by a conventional method.

### Example 11

The compositions containing reduced coenzyme Q10 and a reducing agent after the reaction, which were obtained in Examples 1-9, and the gelatin soft capsule produced in Example 10 were preserved at 25° C. for 1 week in the air. The weight ratio of reduced coenzyme Q10 after the preservation did not decrease from the value at the time of the start of the preservation.

### Comparative Example 1

Limonene (d-limonene, 1.5 g) and each additive (1.50 g) indicated in Table 10 were added in a 25 ml test tube and, in the same manner as in Example 2, oxidized coenzyme Q10 (200 mg, 0.23 mmol) and L-ascorbic acid (245 mg, 6-fold equivalents) as a reducing agent were added, the vessel was substituted with nitrogen, and a reduction reaction was performed by stirring at 80° C. for 16 hr. The weight ratio of reduced coenzyme Q10 in the reaction mixture after the reaction is shown in Table 10. As a comparison, the result by using limonene (3.00 g) and without addition of an additive is also shown in Table 10.

TABLE 10

| | | weight ratio (%) of |
|---|---|---|
| | additive | reduced coenzyme Q10 |
| comparison | without addition | 0 |
| Run. 1 | caprylic acid | 1 |
| Run. 2 | lauric acid | 1 |
| Run. 3 | oleic acid | 0 |

While some of the embodiments of the present invention have been described in detail in the above, it is, however, possible for those of ordinary skill in the art to make various modifications and changes to the particular embodiments shown without substantially departing from the teaching and advantages of the present invention. Such modifications and changes are encompassed in the spirit and scope of the present invention as set forth in the appended claims.

The present invention is based on JP2010-097263 filed in Japan, the contents of which are encompassed in full herein.

1. A method of producing reduced coenzyme Q10 comprising, reducing oxidized coenzyme Q10 with a reducing agent in terpenes in the co-existence of at least one kind of additive selected from the group consisting of alcohols, water, a surfactant and diacylglycerol.

2. The production method according to claim 1, wherein the alcohol is a monovalent alcohol having 1 to 4 carbon atoms or a divalent alcohol having 2 to 4 carbon atoms.

3. (canceled)

4. (canceled)

5. The production method according to claim 1, wherein the terpene is at least one selected from the group consisting of hemiterpene, monoterpene, sesquiterpene, diterpene, sesterterpene and triterpene.

6. The production method according to claim 1, wherein the reducing agent is at least one selected from the group consisting of L-ascorbic acid, D-arabo-ascorbic acid, L-ascorbylpalmitate, L-ascorbylstearate, sodium borohydride, sodium hydrosulfite, retinal, an acerola extract, a pine bark extract, a KOKIYO extract, a gardenia pigment and a KOZU (fragrant vineger).

7. The production method according to claim 1, wherein the reduction reaction is performed in co-existence with fats, oils or mixtures thereof

8. (canceled)

9. The production method according to claim 1, wherein the concentration of oxidized coenzyme Q10 is 0.001 wt % or more relative to total amount of the reaction mixture at the time of the start of the reduction reaction.

10. The production method according to claim 1, wherein the reduction reaction is performed in a preparation.

11. The production method according to claim 10, wherein the preparation is a capsule.

12. The production method according to claim 11, wherein the capsule is a soft capsule.

13. The production method according to claim 1, wherein the reduction reaction is performed under a deoxygenated atmosphere.

14. A composition containing terpenes, a reducing agent, reduced coenzyme Q10 and at least one kind selected from the group consisting of alcohols, water, a surfactant and diacylglycerol.

15. The composition according to claim 14, wherein the alcohol is a monovalent alcohol having 1 to 4 carbon atoms or a divalent alcohol having 2 to 4 carbon atoms.

16. (canceled)

17. The composition according to claim 14, wherein the terpene is at least one selected from the group consisting of hemiterpene, monoterpene, sesquiterpene, diterpene, sesterterpene and triterpene.

18. The composition according to claim 14, wherein the reducing agent is at least one selected from the group consisting of L-ascorbic acid, D-arabo-ascorbic acid, L-ascorbylpalmitate, L-ascorbylstearate, sodium borohydride, sodium hydrosulfite, retinal, an acerola extract, a pine bark extract, a KOKIYO extract, a gardenia pigment and a KOZU (fragrant vineger).

19. The composition according to claim 14, further comprising fats, oils or mixtures thereof.

20. The composition according to claim 14, wherein the content of reduced coenzyme Q10 in the composition is 0.001 wt % or more.

21. The composition according to claim 14, wherein the reduced coenzyme Q10 is added from the outside.

22. The composition according to claim 14, wherein the reduced coenzyme Q10 is reduced in the composition.

23. A method of stabilizing reduced coenzyme Q10, comprising placing terpenes, a reducing agent and at least one kind selected from the group consisting of alcohols, water, a surfactant and diacylglycerol in co-existence.

24. (canceled)

25. (canceled)

26. (canceled)

27. (canceled)

28. (canceled)

29. (canceled)

30. (canceled)
